(19) United States
(12) Patent Application Publication
(10) Pub. No.: US 2014/0025023 A1
(43) Pub. Date: Jan. 23, 2014

(54) WASTE COLLECTION SYSTEM FOR AN EYEWASH STATION

(71) Applicants: Raymond Boissonneault, Upton, MA (US); Kelly Piotti, Rumford, RI (US); James Oberman, East Dubuque, IL (US); Charles Bottieri, Pomfret Center, CT (US)

(72) Inventors: Raymond Boissonneault, Upton, MA (US); Kelly Piotti, Rumford, RI (US); James Oberman, East Dubuque, IL (US); Charles Bottieri, Pomfret Center, CT (US)

(73) Assignee: HONEYWELL INTERNATIONAL, INC., Morristown, NJ (US)

(21) Appl. No.: 13/553,037

(22) Filed: Jul. 19, 2012

(51) Int. Cl. A61F 9/00

(52) U.S. Cl. 604/294

(57) ABSTRACT

An eyewash station includes a body, a door, and a waste collection system for waste eyewash solution. The body contains an eyewash solution source having a defined solution volume. A first area of the door is pivotally connected to the body. An internal surface of a second area of the door has a dispensing member in fluid communication with the eyewash solution source. The second area of the door has a waste collection system including a flexible container, such as a rolled bag. In operation, the eyewash solution is dispensed from the dispensing member and then the waste eyewash solution collected and contained in the rolled bag.

### BACKGROUND

The present application relates generally to an eyewash station, and more particularly, to a waste collection system for an eyewash station to prevent any additional injury to a victim and facilitate cleanup of the waste fluid after the eyewash station has been used.

Eyewash stations use large amounts of eyewash solution to remove contaminants from a victim's eyes. For many stations, the eyewash solution simply spills to the floor and collects at the victim's feet. This can generate multiple hazards to the victim. The fluid immediately becomes a slip hazard in nearly any environment. Additionally, if a station is used in a cold weather environment, the waste eyewash solution could turn into a patch of ice. An additional hazard is created if the eyewash solution is discharged anywhere near electrical systems. This situation is common, as battery charging stations are required to have an eyewash station nearby.

Additionally, the amount of fluid used to flush a victim's eyes generates a large spill on the floor. This takes manpower and time to clean. This is exacerbated, as a large majority of eyewash station activations are not emergency related.

### SUMMARY

An eyewash station includes a body, a door, and a waste collection system for waste eyewash solution. In one embodiment, the eye wash station is gravity-fed. The body contains an eyewash solution source having a defined solution volume. A first area of the door is pivotally connected to the body. An internal surface of a second area of the door has a dispensing member in fluid communication with the eyewash solution source. The second area of the door has a waste collection system that includes a flexible container, such as a rolled plastic bag. The rolled bag has a first end attached to a drain aperture in the second area of the door to collect and contain waste eyewash solution.

The waste collection system further includes a fluid collection area defined proximal to the dispensing member. The fluid collection area defines a drain aperture to allow the exit of waste eye wash solution into the first end of the rolled bag. The first end of the rolled bag is in fluid communication with the drain aperture to collect waste eye wash solution.

The waste collection system further includes a storage compartment with a cover. The storage compartment is located on an exterior surface of the second area of the door and is configured for temporary storage of the rolled bag. The cover is pivotally connected to an open top end of the storage compartment to allow for the release of the rolled bag when the door is inverted for use.

The rolled bag has a defined volume configured for collecting and containing at least the defined volume of eyewash solution dispensed. The rolled bag has a defined length configured for the second end of the rolled bag to engage a floor surface below the eyewash station and to allow the substantial weight of the collected waste eye wash solution to rest on the lower surface. The rolled bag has a wall having a defined thickness to prevent the bag from rupturing while containing the full volume of the defined solution volume while being configured to be tightly rolled in the stored position within the storage compartment.

In operation, when the door is lowered from the upright position to the activated prone position, the cover pivots away from the storage compartment to release the rolled bag to a lower surface. When the solution begins to flow from the dispensing member, the waste solution is temporarily collected and contained in the fluid collection area of the door before exiting through the drain aperture and into the rolled bag.

Accordingly, an objective is to provide an eyewash station that collects and contains waste eyewash solution within a rolled bag.

Other objects, features and advantages shall become apparent as the description thereof proceeds when considered in connection with the accompanying illustrative drawings.

### BRIEF DESCRIPTION OF THE DRAWING FIGURES

An exemplary embodiment will now be described further by way of example with reference to the following examples and figures, which are intended to be illustrative only and in no way limiting upon the scope of the disclosure.

FIG. 1 is a perspective view of an exemplary embodiment of the present eyewash station and waste collection system;

FIG. 2 is a cross-sectional view thereof;

FIG. 3 is a perspective view thereof with the door in the activated prone position;

FIG. 4 is a perspective view thereof with the door in the activated prone position and the waste collection system fully deployed; and

FIG. 5 is a partial cross-sectional view showing the bag in the rolled up position and the deployed position (broken line).

### DETAILED DESCRIPTION OF AN EXEMPLARY EMBODIMENT

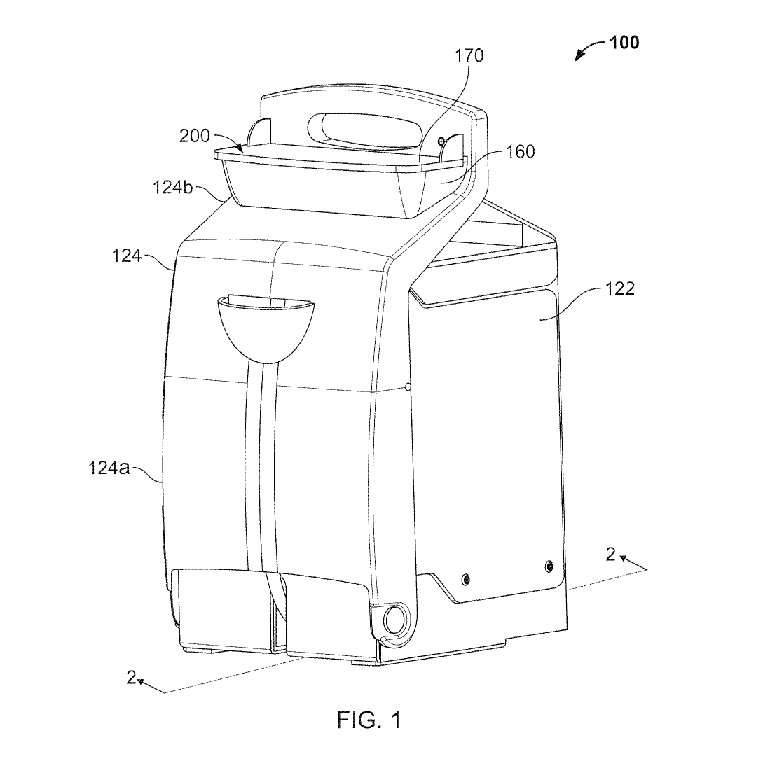

Generally referring to FIGS. 1-5, an exemplary embodiment of the eye wash station 100 with a waste collection system 200 is disclosed. The self-contained, portable eyewash station 100 includes a body 122, one or more doors 124, one or more dispensing members 112, such as a dispensing nozzle, an eyewash solution source 114, such as a solution bag containing eyewash solution 116, a flow conduit 118 for connecting the eyewash solution source 114 and the dispensing member 112, and an improved waste collection system 200 for waste eyewash solution. In one embodiment, the eye wash station 100 is gravity-fed, portable, disposable, and lightweight. The waste collection system 200 for the emergency eyewash station 100 is provided to prevent any additional injury to a victim and facilitate cleanup of the waste eyewash fluid after the eyewash station 100 has been used.

The body 122 or housing assembly contains the eyewash solution source 114 having a defined solution volume. In one embodiment, the eyewash solution source is located above the dispensing member 112 where the head pressure of the eyewash solution source creates a gravity-fed flow of the eyewash solution through the flow conduit 118 to the dispensing member.

A first area 124A of the door 124 is connected to the body 122. In one embodiment, the first area 124A of the door 124 is pivotally connected to the body 122. Of course, the door 124 and body 122 may be connected relative to one other using other means, such as a hinge, or slidably engage one another. An internal surface of a second area 124B of the door 124 has the dispensing member 112 in fluid communication with the eyewash solution source. The dispensing member 112 is located on the door 124. In one embodiment, the dispensing member 112 may have a cover which is removed before the eye wash solution is dispensed.

The second area 124B of the door 124 has the waste collection system 200 including a flexible container 130, preferably a thin, rolled, plastic or plastic film bag. It should be appreciated that other flexible, relatively thin, and lightweight waste collection bags, long film tubes, flexible tubing, and flexible hoses capable of being tightly wound and stored, may be used beyond the preferred rolled plastic film bag. The rolled bag 130 or other flexible containers are relatively lightweight to reduce the overall weight of the eyewash station 100 and therefore may be made of other lightweight, flexible, expandable, or other pliable materials that meet these criteria.

The rolled bag 130 has a first end 130A attached to the second area 124B of the door 124 to collect and contain waste eyewash solution. In one embodiment, the first end 130A or free end of the bag is attached to a flange on the door. In another embodiment, the second end 130B of the flexible container or rolled bag may fluidly connect with other secondary fluid storage devices, such as buckets or other storage containers.

The waste collection system 200 further includes a fluid collection area 140 defined proximal to the dispensing member 112. The fluid collection area 140 defines a drain aperture 150 to allow the exit of waste eye wash solution through the door 124 and into the first end 130A of the rolled bag 130. The fluid collection area 140 funnels the waste eye wash solution to the drain aperture 150. The first end 130A of the rolled bag 130 is in fluid communication with the drain aperture 150 to collect waste eye wash solution. Alternatively, the fluid collection area 150 defines a larger volume to collect a substantial or entire volume of waste eye wash solution dispensed before it is collected and maintained in a flexible container 130.

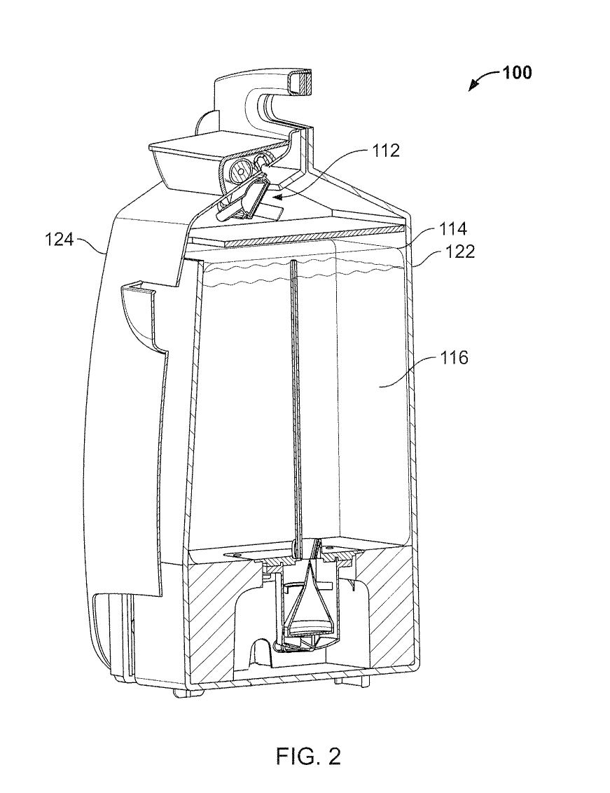

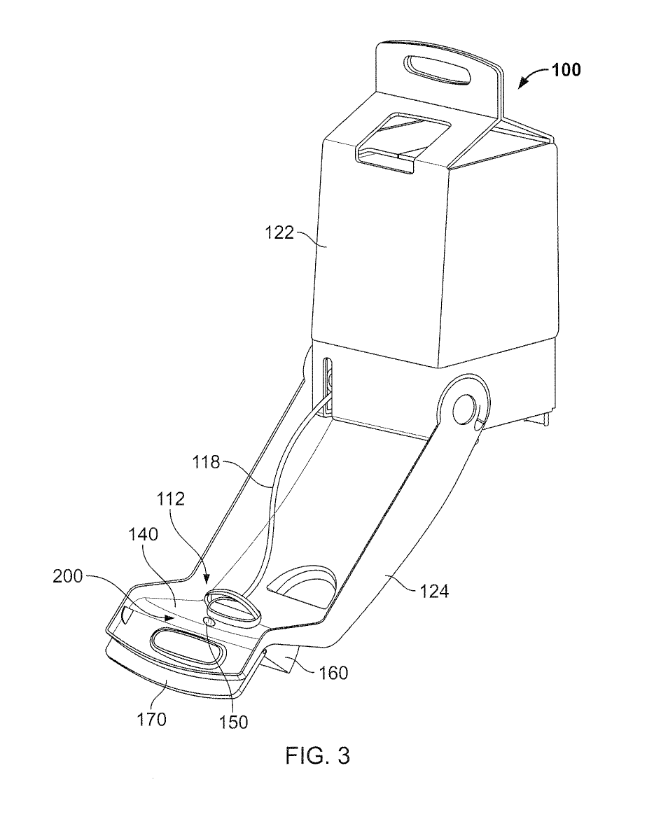

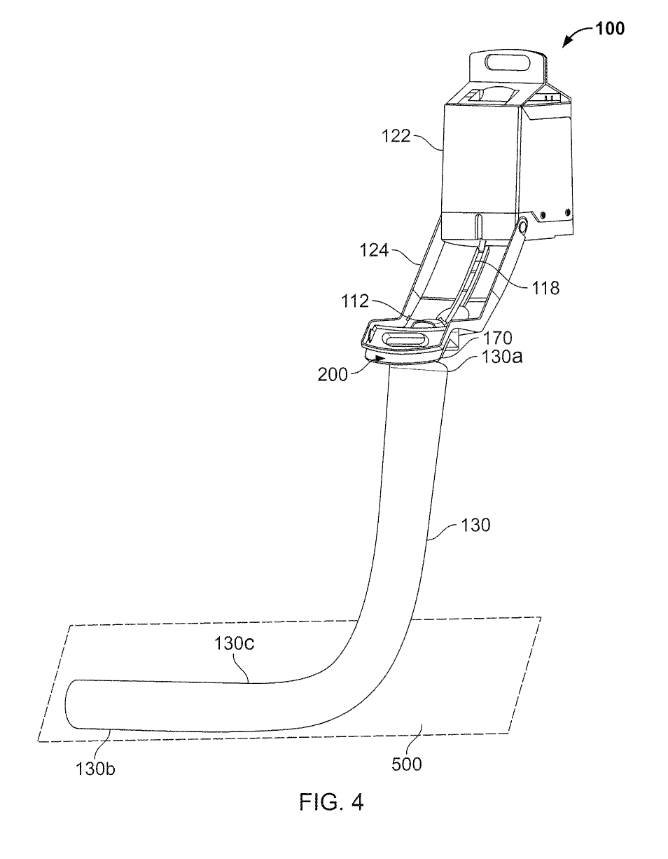

The waste collection system 200 further includes a storage compartment 160 with a cover 170 connected thereto. The storage compartment 160 is located on an exterior surface of the second area 124B of the door and configured for temporary storing of the rolled bag 130. The cover 170 is pivotally connected to an open top end of the storage compartment 160 to allow for the release of the rolled bag 130. When the door 124 is in the upright position (FIGS. 1-2), the cover 170 over the compartment 160 protects the bag 130 and restricts the bag 130 from unrolling. The cover 170 is configured so that it will automatically open when the door 124 is moved to the activated prone position (FIG. 3). When the door 124 is moved to the activated prone position, gravity causes the cover 170 to open and the rolled bag 130 to drop down and unroll to a lower surface 500 (FIG. 4), such as a floor. Of course, the lower surface may be any surface beneath or below the eye wash station 100.

In one embodiment, the rolled bag 130 has a defined volume configured for collecting and containing at least the defined volume of eyewash solution dispensed. The rolled bag 130 has a defined length configured for the second end 130B of the rolled bag 130 to engage the lower surface 500 positioned along an axis lower than the eyewash station 100. The rolled bag 130 has a defined length configured to facilitate at least the substantial weight of the collected waste eye wash solution to rest on the lower surface 500. In one embodiment, the rolled bag 130 or other flexible container engages the lower surface 500 so that no significant force is transferred to the door of the eye wash station. The rolled bag 130 has a defined length to accommodate the defined solution volume in the portion of the bag 130C that is resting on the lower surface 500. Alternatively, support straps (not shown) may be used to support the weight of the bag 130 containing waste solution.

In one embodiment, the rolled bag 130 has a wall having a defined thickness to prevent the bag 130 from rupturing while containing the full volume of the defined solution volume while also being configured to be tightly rolled in the stored position within the storage compartment 160. As a result, the tightly rolled bag 130, as part of the waste collection system 200, is significantly more compact and portable.

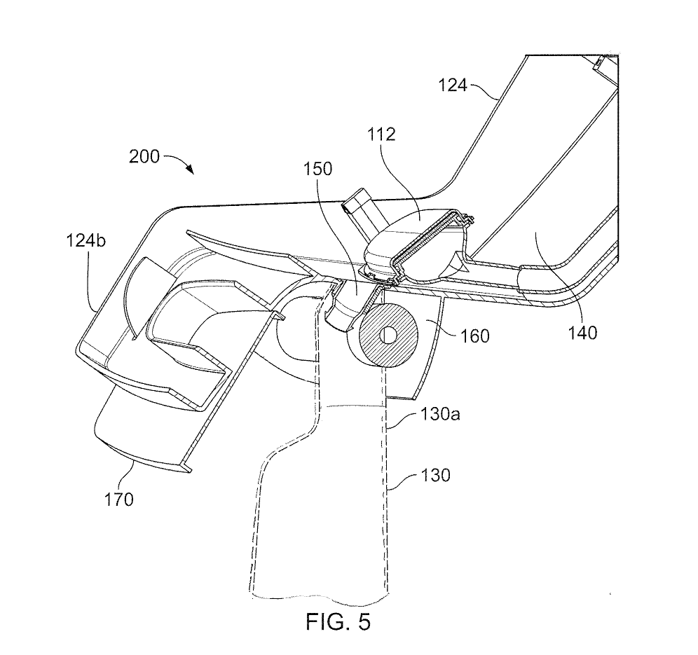

In operation, when the door 124 is pivoted from the upright position (FIG. 2) to the activated prone position (FIG. 5), the cover 170 pivots away from the storage compartment 160 to release the rolled bag 130 to engage the lower surface 500. When the solution begins to flow from the dispensing member 112 due to head pressure, the waste solution is temporarily collected and contained in the fluid collection area 140 of the door 124 before exiting through the drain aperture 150 and into the rolled bag 130.

Accordingly, an objective is to provide an eyewash station 100 that collects and contains waste eyewash solution within a flexible container 130, such as a rolled plastic or plastic film bag.

For these reasons, the present eyewash station 100 is believed to represent a significant advancement in the art, which has substantial commercial merit.

While there is shown and described herein certain specific structure embodying the eyewash station, it will be manifest to those skilled in the art that various modifications and rearrangements of the parts may be made without departing from the spirit and scope of the underlying concepts and that the same is not limited to the particular forms or embodiments herein shown and described except insofar as indicated by the scope of the appended claims.

1. An eyewash station, comprising:
a body containing an eyewash solution source having a defined solution volume;
a door connected to the body, the door including a dispensing member in fluid communication with the eyewash solution source; and
a waste collection system located within the door, the waste collection system includes a flexible container connected to the door to collect and contain waste eyewash solution, whereby the flexible container is offset from the body during operation.

2. The eyewash station of claim 1, wherein the waste collection system includes a fluid collection area on the door and defined proximal to the dispensing member.

3. The eyewash station of claim 2, further wherein the fluid collection area defines a drain aperture to allow the exit waste solution into a first end of the flexible container.

4. The eyewash station of claim 3, further comprising a storage compartment located on an exterior surface of the door, said storage compartment being configured for temporary storage of the flexible container.

5. The eyewash station of claim 4, further comprising a cover connected to the storage compartment.

6. The eyewash station of claim 5, wherein a first end of the flexible container is in fluid communication with the drain aperture to collect waste eye wash solution.

7. The eyewash station of claim 1, wherein the flexible container has a defined volume configured for collecting and containing at least the defined solution volume of eyewash solution dispensed.

8. The eyewash station of claim 1, wherein the flexible container has a defined length configured for a second end of the flexible container to engage a lower surface beneath the eyewash station.

9. The eyewash station of claim 1, wherein the flexible container has a defined length to accommodate the defined solution volume in the portion of the flexible container that is resting on the lower surface.

10. The eyewash station of claim 1, wherein the flexible container is configured with a wall having a defined thickness to prevent the flexible container from rupturing while being handled with the full volume of the defined solution volume while being configured to be tightly rolled in the stored position within the storage compartment.

11. The eyewash station of claim 1, wherein the eyewash station is gravity-fed portable, and lightweight.

12. The eyewash station of claim 1, wherein the flexible container is a rolled bag.

13. The eyewash station of claim 12, wherein the rolled bag is an elongated plastic tube.

14. The eyewash station of claim 1, wherein the body is pivotally connected to a first area of the door.

15. The eyewash station of claim 5, wherein the cover is pivotally connected to the storage compartment.
